(19) United States
(12) Patent Application Publication
(10) Pub. No.: US 2013/0072738 A1
(43) Pub. Date: Mar. 21, 2013

(54) SUPPORTED CATALYST FOR DIRECT DEHYDROGENATION OF n-BUTANE AND PREPARING METHOD OF BUTENES FROM n-BUTANE USING THE SAME

(71) Applicant: Korea Institute of Science and Technology, Seoul (KR)

(72) Inventors: Kwang Deog JUNG, Seoul (KR); Oh Shim JOO, Seoul (KR); Jun Woo OH, Seoul (KR); Shashikala VELDRUTHI, Seoul (KR)

(73) Assignee: KOREA INSTITUTE OF SCIENCE AND TECHNOLOGY, Seoul (KR)

(21) Appl. No.: 13/623,313

(22) Filed: Sep. 20, 2012

(30) Foreign Application Priority Data
Sep. 20, 2011 (KR) 10-2011-0094777

(51) Int. Cl. B01J 23/72; C07C 5/333

(52) U.S. Cl. 585/660; 502/331

(57) ABSTRACT

Disclosed is a method for preparing butene from n-butane by direct dehydrogenation using a specific supported catalyst.

When the supported catalyst of the present invention, wherein platinum, palladium or platinum and palladium as main catalyst and a copper cocatalyst are supported on an alumina support, is used to prepare n-butenes by direct dehydrogenation of n-butane, production of byproducts resulting from cracking and isomerization, deposition of carbon and inactivation of the catalyst are effectively suppressed and conversion ratio of n-butane and selectivity to n-butenes are remarkably improved.

### CROSS-REFERENCE TO RELATED APPLICATIONS

This application claims priority under 35 U.S.C. §119 to Korean Patent Application No. 10-2011-0094777, filed on Sep. 20, 2011, in the Korean Intellectual Property Office, the disclosure of which is incorporated herein by reference in its entirety.

### BACKGROUND

(a) Technical Field

The present invention relates to a novel supported catalyst useful for direct dehydrogenation of n-butane and a method for preparing n-butenes from n-butane using the catalyst.

(b) Background Art

1,3-Butadiene is an important chemical compound in the field of petrochemistry. For example, it is used as a raw material for preparation of a butadiene homopolymer, a synthetic rubber such as styrene-butadiene rubber (SBR) or nitrile rubber or a thermoplastic terpolymer such as acrylonitrile-butadiene-styrene copolymer (ABS). In China, in particular, the demand on the acrylonitrile-butadiene-styrene copolymer has been increasing remarkably with the growth of its electronics market. And in Korea, the demand on 1,3-butadiene has been exceeding its supply with the increased production of the styrene-butadiene rubber.

More than 90% of butadiene supplied to the petrochemical market is prepared by extraction of C₄ hydrocarbons through naphtha cracking. However, since the naphtha cracking process produces not only 1,3-butadiene but also methane, ethane, ethene, acetylene, propane, propene, propyne, allene, butene, butadiene, butyne, methylallene and C₅ or higher hydrocarbons, the yield of 1,3-butadiene is not high. Since it is difficult to meet the gradually increasing demand on 1,3-butadiene and to effectively cope with the change in market situations with the method of preparing 1,3-butadiene by naphtha cracking, a process of preparing 1,3-butadiene by dehydrogenation of C₄ hydrocarbons produced by naphtha cracking was developed.

Although the production of 1,3-butadiene was increased by the process of producing butadiene from C₄ raffinate, this process is directly affected by oil price since the C₄ raffinate used as reactants in the dehydrogenation are obtained from crude oil. If the oil price remains persistently high, the cost of 1,3-butadiene will also increase and its supply may be restricted. For this reason, there is a need to prepare 1,3-butadiene from chemical sources other than crude oil. A process of preparing n-butenes from dehydrogenation of n-butane obtained from coal or natural gas is drawing attentions as an alternative. The process of producing n-butene (1-butene, trans-2-butene and cis-2-butene) from dehydrogenation of n-butane and then obtaining 1,3-butadiene by oxidatively dehydrogenating again the butene is advantageous in that valuable products can be produced at low cost without being affected by oil price.

Dehydrogenation of n-butane is achieved either by oxidative dehydrogenation or by direct dehydrogenation. Since carbon monoxide and carbon dioxide are produced together in oxidative dehydrogenation of n-butane, the selectivity of the major product, i.e., butene (1-butene, trans-2-butene and cis-2-butene), decreases. In direct dehydrogenation of n-butane, n-butenes and hydrogen are produced from catalytic reaction of n-butane. Although the direct dehydrogenation is advantageous in that selectivity is higher than that oxidative dehydrogenation, catalytic activity decreases owing to deposition of carbon. Hence, researches are under way to improve the yield of n-butenes by using a catalyst capable of avoiding carbon deposition and exhibiting high selectivity.

Noble metal catalysts such as platinum are commonly used for direct dehydrogenation of n-butane. However, since it is impossible to control the many side reactions such as deposition of carbon, degradation of hard paraffin, isomerization, aromatization, oligomerization, etc. only with platinum, various cocatalysts and supports are being studied. The supports for platinum-based catalysts known to be effective for preparation of butene by direct dehydrogenation of n-butane include an alumina support [McNamara, J. M. Jackson, S. D. Lennon, Catal. Today vol. 81, 583 (2003)], a spinel-based support synthesized by adding magnesium or zinc to alumina to decrease the acidity of alumina [S. Bocanegra, A. Ballarini, P. Zgolicz, O. Scelza, S. de Miguel, Catal. Today vol. 143, 334 (2009)], a support prepared by adding an alkali metal to alumina such as sodium-alumina [S. de Miguel, S. Bocanegra, J. Vilella, A. Guerrero-Ruiz, O. Scelza, Catal. Lett. vol. 119, 5 (2007)], a support synthesized from alumina and zirconium [C. Larese, J. M. Campos-Martin, J. L. G. Fierro, Langmuir vol. 16, 10294 (2000)], and the like. For cocatalysts, tin which reduces the adsorption energy of coke precursors adsorbed on the surface of platinum [A. Bocanegra, S. R. de Miguel, A. A. Castro, O. A. Scelza, Catal. Lett. vol. 96, 129 (2004)], an alkali metal cocatalyst such as potassium or lithium which decreases acidity of the catalytic surface [Liu Jinxiang, Gao Xiuying, Zhang Tao, Lin Liwu, Thermochim. Acta vol. 179, 9 (1991)], etc. are mainly used.

The inventors of the present invention have studied consistently in order to develop a method capable of suppressing deactivation of catalyst due to deposition of carbon even at high temperatures and preventing side reactions such as cracking, isomerization, aromatization, oligomerization, etc. while providing better conversion of n-butane as compared to the existing art.

As a result, they have found out that when a supported catalyst in which a main catalyst such as platinum, palladium or platinum and palladium and a copper cocatalyst are supported on an alumina support is used as a catalyst for direct dehydrogenation of n-butane, a superior catalytic activity can be achieved and a higher conversion of n-butane can be maintained as compared to the existing platinum-supported alumina catalyst (Pt/alumina) even at high temperatures since carbon deposition and side reactions are prevented.

### SUMMARY

The present invention is directed to providing a novel supported catalyst for direct dehydrogenation of n-butane.

The present invention is also directed to providing a method for preparing n-butenes by direct dehydrogenation of n-butane using the supported catalyst.

In an aspect, the present invention provides a catalyst for direct dehydrogenation of n-butane in which a main catalyst comprising platinum, palladium or platinum and palladium and a copper cocatalyst are supported on an alumina support.

In an aspect, the present invention provides a method for preparing n-butenes by carrying out direct dehydrogenation of n-butane in the presence of the supported catalyst.

The supported catalyst of the present invention exhibits superior catalytic activity when used for direct dehydrogenation of n-butane.

The supported catalyst of the present invention results in less deactivation since the production of hydrocarbon byproducts and deposition of carbon in the catalyst bed are prevented.

The supported catalyst of the present invention increases conversion of n-butane and selectivity to n-butenes (1-butene, trans-2-butene and cis-2-butene).

Accordingly, the supported catalyst of the present invention may be usefully applied to produce n-butenes in large scale by direct dehydrogenation of n-butane.

The above and other aspects and features of the present invention will be described infra.

Other features and aspects of the present invention will be apparent from the following detailed description, drawings and claims.

### BRIEF DESCRIPTION OF THE DRAWINGS

The above and other objects, features and advantages of the present invention will now be described in detail with reference to certain exemplary embodiments thereof illustrated in the accompanying drawings which are given hereinbelow by way of illustration only, and thus are not limitative of the invention, and wherein:

FIG. 1 shows conversion of n-butane and selectivity to n-butenes with reaction time of direct dehydrogenation in the presence of a platinum-palladium-copper/alumina catalyst of the present invention; and

FIG. 2 shows conversion of n-butane and selectivity to n-butenes with reaction time of direct dehydrogenation in the presence of a platinum-copper/alumina catalyst of the present invention at reaction temperatures of 500° C., 525° C. and 550° C.

### DETAILED DESCRIPTION

Hereinafter, reference will now be made in detail to various embodiments of the present invention, examples of which are illustrated in the accompanying drawings and described below. While the invention will be described in conjunction with exemplary embodiments, it will be understood that the present description is not intended to limit the invention to those exemplary embodiments. On the contrary, the invention is intended to cover not only the exemplary embodiments, but also various alternatives, modifications, equivalents and other embodiments, which may be included within the spirit and scope of the invention as defined by the appended claims.

The present invention relates to a supported catalyst used for preparation of n-butenes from n-butane by direct dehydrogenation. In the supported catalyst, a main catalyst comprising platinum, palladium or platinum and palladium and copper as a cocatalyst component are supported on an alumina support. Whereas copper is used as an additive for modifying the support in the prior art, copper is supported as a cocatalyst component in the supported catalyst of the present invention.

The alumina used as the support of the supported catalyst of the present invention may be one commonly used as a support in the related art. Specifically, at least one selected from the group consisting of γ-alumina, η-alumina, α-alumina, κ-alumina and θ-alumina may be used. More specifically, the support may comprise θ-alumina. The alumina support may be prepared by dissolving an aluminum precursor such as aluminum nitride hydrate in water, precipitating alumina by titrating with an alkaline solution and performing hydrothermal synthesis, followed by drying and baking.

In the supported catalyst of the present invention, the platinum, palladium or platinum and palladium used as the main catalyst is supported in an amount of 0.1-5.0 wt %, specifically 0.2-2.0 wt %, based on the weight of the supported catalyst. If the supporting amount of the main catalyst is too small, catalytic activity may be very low. And, if the supporting amount of the main catalyst is too large, production cost increases since the expensive noble metal is used in excessive amount and catalytic activity may decrease on the contrary. Accordingly, the aforesaid range is appropriate. A precursor used to support the main catalyst maybe a compound containing platinum or palladium. For example, oxide, hydroxide, chloride, hydrochloride, phosphate, nitrate, acetate, acetylacetonate, etc. of these metals may be used.

In the supported catalyst of the present invention, the copper used as the cocatalyst is supported in an amount of 0.1-5.0 wt %, specifically 0.2-2.0 wt %, based on the weight of the supported catalyst. If the supporting amount of the copper cocatalyst is too small, the cocatalytic effect of copper may not be exhibited. And, if the supporting amount is too large, dispersibility of the platinum or palladium used as the main catalyst may decrease. A precursor used to support the cocatalyst maybe a compound containing copper. For example, oxide, hydroxide, chloride, hydrochloride, phosphate, nitrate, acetate, acetylacetonate, etc. of copper may be used.

A method for preparing the supported catalyst according to the present invention descried above will be described in detail.

First, an alumina support is prepared.

An alumina precursor is dissolved in distilled water. After adjusting pH to 6-9 by adding a basic precipitant, hydrothermal synthesis is performed at 150-200° C. to prepare alumina. The basic precipitant may be ammonia water, sodium hydroxide, or the like. The prepared alumina is dried at 80-110° C. and baked at 500-700° C. under air atmosphere to prepare an alumina support.

Then, a main catalyst and a cocatalyst are supported on the alumina support to prepare a supported catalyst of the present invention.

A precursor of platinum or palladium used as the main catalyst and a precursor of copper used as the cocatalyst are respectively dissolved in ethanol, supported on the alumina support in an amount described above, and then uniformly mixed. Thereafter, the supported catalyst of the present invention is prepared by drying at 90-130° C. and baking in a baking furnace at 400-1000° C., specifically at 500-800° C., in the air. If the baking temperature is too low, the salt included in the metal precursor may remain unremoved. And, if the baking temperature is too high, catalyst structure may be altered. Hence, the aforesaid baking temperature range is appropriate.

n-Butenes are prepared by direct dehydrogenation of n-butane using the prepared supported catalyst.

The direct dehydrogenation in accordance with the present invention may be performed by loading the catalyst in a linear reactor and continuously passing a reactant through a catalyst bed in the reactor while maintaining the reaction temperature of the catalyst bed constant. The reaction temperature may be adjusted to 500-650° C., specifically 550-625° C. If the reaction temperature is too low, dehydrogenation may not occur well because the catalyst is not activated. And, if the reaction temperature is too high, side reactions such as cracking, isomerization, aromatization, oligomerization, etc. may increase. The reactant is a mixture gas of n-butane and hydrogen, at a volume ratio of 1:0.1-3.0, specifically 1:0.3-2.0, more specifically 1:0.5-1.2. If the mixing ratio of n-butane and hydrogen in the reactant is outside the aforesaid range, the deactivation of the catalysts is severe in the mixture gas with low hydrogen content and thermodynamic activity may decrease in the mixture gas with high hydrogen content. And, the reactant may be injected at a space velocity of 1,000-50,000 h⁻¹, specifically 3,000-20,000 h⁻¹, on the basis of n-butane. If the space velocity is lower than 1,000 h⁻¹, profitability may decrease since production per unit time is too small. And, if it exceeds 50,000 h⁻¹, yield of butadiene may decrease since the time for the n-butene to react with the catalyst is short.

When n-butane is directly dehydrogenated using the supported catalyst of the present invention, n-butenes can be prepared with high conversion of n-butane and selectivity to n-butenes. The n-butane used as the reactant in the present invention may be obtained from coal or natural gas. The n-butenes prepared by the present invention may be usefully used for preparation of 1,3-butadiene.

### EXAMPLES

The present invention will be described in more detail through examples. The following examples are for illustrative purposes only and it will be apparent to those skilled in the art not that the scope of this invention is not limited by the examples.

### Example 1

### Preparation of Supported Catalyst

Aluminum nitrate nonahydrate (Al(NO₃).9H₂O), which was used as an aluminum precursor, was dissolved in distilled water. After stirring for 30 minutes using a magnetic stirrer to obtain a sample of uniform composition, alumina was precipitated by adding 28-30% ammonia water diluted with water (1:1) dropwise until pH 7. After stirring for 20 hours using a magnetic stirrer, the resulting solution was put in a high-pressure reactor and subjected to hydrothermal synthesis for 20 hours using an agitated dryer at 190° C. The precipitated solution was filtered through a vacuum filter and the collected solid sample was dried at 110° C. for 16 hours. The dried solid sample was heat-treated in an electric furnace maintained at 600° C. under air atmosphere for 5 hours to prepare an alumina support. The specific surface area of the prepared support was 151 m²/g.

Chloroplatinic acid hexahydrate (H₂PtCl₆.6H₂O) was used as a platinum precursor, palladium nitrate (Pd(NO₃)₂.xH₂O) as a palladium precursor, and copper nitrate (Cu(NO₃)₂.xH₂O) as a copper precursor. The precursors were weighed, dissolved in ethanol at an appropriate ratio, and supported on the alumina support prepared above, with the desired catalyst weight ratio. To obtain a sample of uniform composition, the solution was stirred with a glass rod at room temperature until drying. The as-dried sample was dried again at 110° C. for about 16 hours and the resulting solid sample was baked in an electric furnace maintained at 600° C. for 4 hours under air atmosphere.

As a result, platinum-copper/alumina, palladium-copper/alumina and platinum-palladium-copper/alumina catalysts were prepared. Each catalyst was denoted as aPt-bPd-cCu/Al₂O₃ (where a, b and c are supporting amounts (wt %) of the respective metals based on the total weight of the supported catalyst).

### Example 2

### Direct Dehydrogenation of n-butane Using Supported Catalyst

Activity of the supported catalyst prepared in Example 1 was determined as follows.

The supported catalyst (1 g) was fixed in a stainless steel linear reactor. While maintaining the temperature of the catalyst bed at 550° C., direct dehydrogenation was performed by supplying a reactant comprising n-butane, hydrogen and nitrogen (1:1:1, based on volume) to the reactor at a space velocity (GHSV) of 6,000 h⁻¹, on the basis of n-butane.

The reaction product was analyzed by gas chromatography equipped with a flame ionization detector. Conversion ratio of n-butane, selectivity for butene and yield were calculated according to Equations 1-3.

Conversion ratio of n-butane (%)=(Moles of reacted n-butane)/(Moles of supplied n-butane)×100 [Equation 1]

Selectivity for butene (%)=(Moles of produced butene)/(Moles of reacted n-butane)×100 [Equation 2]

Yield of butene (%)=(Moles of produced butene)/(Moles of supplied n-butane)×100 [Equation 3]

The result of carrying out direct dehydrogenation of n-butane at 500° C. using 1Pd/Al₂O₃, 1Pd-1Cu/Al₂O₃, 1Pd-2Cu/Al₂O₃ and 1Pd-3Cu/Al₂O₃ supported catalysts is shown in Table 1.

TABLE 1

| | Conversion | Selectivity²⁾ (%) | | | | | |
|---|---|---|---|---|---|---|---|
| Catalyst | ratio¹⁾ (%) | Cracking | Isobutane | Isobutene | n-Butene³⁾ | Butadiene | Others |
| 1Pt/Al₂O₃ | 24.2/19.3 | 35.1/16.2 | 4.0/1.9 | 4.6/3.0 | 53.0/76.8 | 0.8/1.0 | 2.6/1.2 |
| 1Pd/Al₂O₃ | 5.8/3.8 | 16.5/13.4 | 1.8/2.9 | 7.8/6.1 | 70.5/75.6 | 0.6/0.3 | 2.8/1.7 |
| 1Pd—1Cu/Al₂O₃ | 12.5/12.6 | 4.0/2.4 | 1.4/1.1 | 4.1/2.5 | 88.2/93.1 | 1.0/0.4 | 1.4/0.5 |
| 1Pd—2Cu/Al₂O₃ | 13.4/16.0 | 4.0/2.8 | 1.2/1.0 | 3.5/2.1 | 88.6/92.7 | 1.4/0.8 | 1.3/0.5 |
| 1Pd—3Cu/Al₂O₃ | 13.1/14.4 | 4.3/3.0 | 1.2/1.0 | 4.2/2.5 | 87.7/92.3 | 1.2/0.5 | 1.5/0.7 |

¹⁾Conversion ratio: A/B (A: conversion ratio measured after reaction for 10 minutes, B: conversion ratio measured after reaction for 300 minutes)
²⁾Selectivity: A/B (A: selectivity measured after reaction for 10 minutes, B: selectivity measured after reaction for 300 minutes)
³⁾Selectivity for butene: selectivity for total butene including 1-butene, isobutene, trans-2-butene and cis-2-butene

As seen from Table 1, although the catalytic activity of 1Pt/Al₂O₃ was initially high with a conversion ratio of butane of 24.2%, the conversion ratio of butane decreased rapidly to 19.3% after reaction for 300 minutes and the selectivity for n-butene was very low.

The 1Pd/Al₂O₃ catalyst showed initially poor catalytic activity, with a very low conversion ratio of butane of 5.8%. In contrast, the palladium-copper/alumina catalyst in which copper was further supported as a cocatalyst showed more than 2 timed improved catalytic activity as compared to the palladium alumina catalyst and the selectivity for n-butene was maintained above 90% even after reaction for 300 minutes. This suggests the addition of copper prevents inactivation of the catalyst by minimizing carbon deposition.

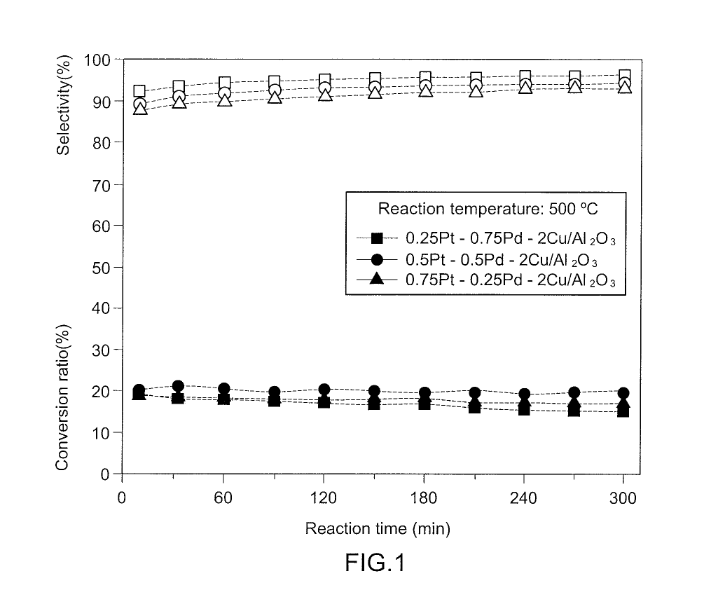

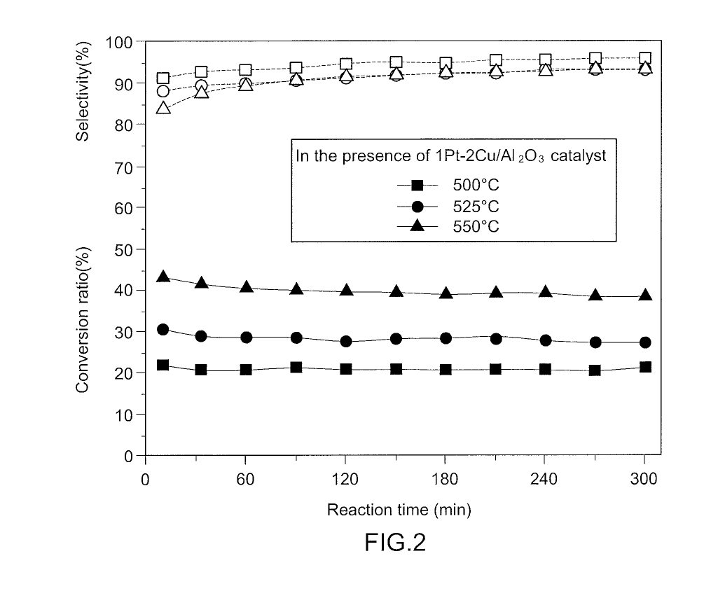

FIG. 1 shows the result of performing direct dehydrogenation of n-butane at 500° C. using 0.25Pt-0.75Pd-2Cu/ Al₂O₃, 0.5Pt-0.5Pd-2Cu/ Al₂O₃ and 0.75Pt-0.25Pd-2Cu/ Al₂O₃ catalysts. And, FIG. 2 shows the result of performing direct dehydrogenation of n-butane at 500, 525 and 550° C. using the 1Pt-2Cu/Al₂O₃ catalyst.

From FIG. 1 and FIG. 2, it can be seen that the platinum-palladium-copper/alumina catalyst and the platinum-copper/alumina catalyst according to the present invention maintain conversion ratio of butane and selectivity for butene even after reaction for 300 minutes. That is to say, whereas the existing platinum/alumina catalyst exhibits initially high catalytic activity but rapidly decreased catalytic activity after reaction for 300 minutes, the catalysts according to the present invention wherein copper was further added as cocatalyst in addition to the platinum main catalyst are not deactivated.

Since the platinum-palladium-copper/alumina catalyst showed no significant difference from the platinum-copper/alumina catalyst in terms of conversion ratio and selectivity, a catalyst can be prepared more economically by partially replacing the expensive noble metal platinum with palladium.

As described above, the supported catalyst of the present invention may be usefully applied to produce butene in large scale by direct dehydrogenation of n-butane.

What is claimed is:

1. A catalyst for direct dehydrogenation of n-butane, which is a supported catalyst in which a main catalyst comprising platinum, palladium or platinum and palladium and a copper cocatalyst are supported on an alumina support.

2. The catalyst for direct dehydrogenation of n-butane according to claim 1, wherein the main catalyst is supported in an amount of 0.1-5.0 wt % and the cocatalyst is supported in an amount of 0.1-5.0 wt % based on the weight of the supported catalyst.

3. The catalyst for direct dehydrogenation of n-butane according to claim 1, wherein alumina support comprises at least one of γ-alumina, η-alumina, α-alumina, κ-alumina and θ-alumina.

4. A method for preparing n-butenes by performing direct dehydrogenation of n-butane in the presence of the catalyst for direct dehydrogenation of n-butane according to any one of claims 1 to 3.

5. The method for preparing n-butenes according to claim 4, wherein the direct dehydrogenation is performed at 500-650° C., with the volume ratio of n-butane and hydrogen used as reactant maintained at 1:0.1-3.0 and the reactant being supplied at a space velocity of 1,000-50,000 h⁻¹, on the basis of n-butane.
